(19) United States
(12) Patent Application Publication
(10) Pub. No.: US 2007/0161971 A1
(43) Pub. Date: Jul. 12, 2007

(54) Urinary catheterization kit

(76) Inventor: Jamie Glen House, Colorado Springs, CO (US)

Correspondence Address: CONLEY ROSE, P.C., P. O. BOX 3267, HOUSTON, TX, 77253-3267, US

(21) Appl. No.: 11/326,699

(22) Filed: Jan. 6, 2006

(51) Int. Cl. A61M 27/00

(52) U.S. Cl. 604/544; 206/364

(57) ABSTRACT

A urinary catheter assembly is disclosed that comprises a catheter and pliable sheath, which protects at least part of the urethra-insertable portion of the catheter without enclosing the urine outlet of the catheter. In certain embodiments a free end of the catheter is made available to facilitate manipulation of the outlet for collection of a urine specimen. The sheath is attached to the catheter so as to prevent urine from entering the sheath and contaminating the catheter. A catheterization kit containing the catheter assembly is also disclosed, in which several items of equipment from a conventional catheterization kit are obviated, allowing for decreased nursing catheterization time and reduction of risk of urinary tract infection in the patient.

### CROSS-REFERENCE TO RELATED APPLICATIONS

N/A

### STATEMENT REGARDING FEDERALLY SPONSORED RESEARCH OR DEVELOPMENT

N/A

### BACKGROUND OF THE INVENTION

1. Technical Field of the Invention

The present invention relates to devices and methods for catheterization of the urinary bladder.

2. Description of Related Art

In hospital settings today, it is commonplace for nurses to perform catheterization procedures using convenience packs, or pre-assembled kits, which typically contain a vinyl or red latex rubber catheter, waterproof absorbent underpad, fenestrated drape, disposable gloves, a sealed packet containing about 22.5 mL of Povidone-Iodine solution, five prepping cotton balls in a disposable tray compartment, a sealed packet containing sterile lubricating jelly, a plastic forceps, a sterile specimen bottle, and a 1000 mL graduated basin. All of these items are packaged together and sterilized. In practice, a nurse opens the tray, dons sterile gloves and places the drape around the patient's genitalia. The Povidone-Iodine packet is opened and poured over the five cotton balls. The packet of lubricating jelly is opened and squeezed onto a sterile field. The patient's urethral opening is cleansed with the five saturated cotton balls, holding each with the forceps. The nurse then runs the tip end of the catheter, comprising the first inch or two (about 2.5 cm to about 5 cm) of the insertable portion, through the lubricating jelly. The catheter is then inserted into the urethra and advanced until urine begins to flow. The urine is drained into the receptacle. A urine specimen is caught in the collection container, if needed. The catheter is then removed and all items of equipment are discarded. Care must be taken to maintain sterile procedure, to reduce the risk of urinary tract infection. Because multiple steps are involved in the procedure, a nurse typically spends a significant amount of time (e.g., 10-15 minutes) carrying out each catheterization. This basic procedure is used in virtually every inpatient hospital around the world, and has remained essentially the same for 50 years.

The healthcare industry would welcome a way to simplify and streamline inpatient catheterization procedures and eliminate steps that may compromise sterile technique.

### SUMMARY OF THE PREFERRED EMBODIMENTS

The representative embodiments disclosed herein seek to overcome some of the drawbacks inherent in the prior art by providing devices, kits and methods for simplifying and improving inpatient catheterization equipment and procedures. Accordingly, in certain embodiments, a urinary catheter assembly is provided which comprises a catheter having a urethra-insertable portion and a non-insertable portion. The insertable portion comprises a first end and an insertion stop location adjacent to the non-insertable portion, wherein the non-insertable portion comprises a second end having a urine outlet. The assembly also includes a pliable sheath, or thin plastic bag, comprising a lumen, and enclosing all or part of the insertable portion of the catheter, and has a terminus that is attached to the catheter at an attachment point disposed between the insertion stop location and the outlet end of the catheter, to form a water-tight seal. In some embodiments the attachment point coincides with the outlet end of the catheter. The sheath lumen and the urine outlet of the catheter are prevented from mutual fluid communication, so as to prevent urine from entering the sheath interior or lumen, and to prevent urine from contacting the insertable portion of the catheter.

In certain embodiments, the catheter assembly further comprises a catheter tip guard or introducer that is attached to the opposite end of the sheath. Together the sheath and catheter tip guard, or the like, in cooperation, completely enclose the urethra-insertable portion of the catheter. In certain embodiments, at least part of the non-insertable portion of the catheter comprises an uncovered region of the catheter, which extends outside of the sheath. Thus leaving a exposed portion or “free end” of the catheter, including the urine outlet. The free end facilitates collection of urine specimens and facilitates drainage of urine into a urine receptacle. In some embodiments, the catheter assembly comprises a urine receptacle attached to the non-insertable portion of the catheter. In certain embodiments, the urine receptacle includes a urine drainage tube for conveniently obtaining a sterile urine specimen.

In some embodiments, the free end comprises substantially all of the non-insertable portion of the catheter. In certain embodiments, the sheath attachment point is spaced apart from the outlet end of the catheter a distance of no more than about ⅓ of the total length of the catheter, wherein the ⅓ distance comprises the free end or uncovered region of the catheter.

In some embodiments, the attachment point coincides with the outlet end of the catheter, or is spaced apart from the outlet end a distance (d) of no more than about 2 mm. Even a short length of exposed catheter outside of the sheath can serve as a useful lip for resting on the opening of a small container, to facilitate collection of a urine specimen.

In certain embodiments, the attachment point is spaced apart from the catheter outlet end a distance (d) in the range of about 2 mm to about 50 mm, preferably in the range of about 10 mm to about 30 mm.

In certain embodiments, the sheath terminus is sealingly attached to the attachment point on the catheter.

In certain embodiments, a catheter assembly is provided in which the sheath terminus is slidingly attached to the attachment point on the catheter.

In certain embodiments, a catheter assembly is provided in which an extension is attached to the sheath terminus, wherein the extension comprises a urine spout, to facilitate urine collection. In certain embodiments, the extension comprises a band attached to the sheath terminus at the attachment point.

In accordance with certain embodiments of the present invention, a catheterization kit is provided which comprises an above-described urinary catheter assembly in a sanitary wrapper. In certain embodiments, the kit comprises an above-described urinary catheter assembly, at least one antiseptic swab (e.g., Betadine, Povidone-Iodine), disposable gloves, a urine specimen container, and a tray capable of holding the aforesaid items. The tray is also capable of serving as a urine receptacle. The sanitary wrapper encloses all of the items in the kit.

In certain embodiments, the kit consists essentially of the aforementioned items. In certain embodiments, the kit specifically excludes one or more of the following items which are customary components of conventional catheterization trays: cotton balls, forceps, antiseptic liquid packet, lubricant gel packet, one or more drapes, and an absorbent sheet.

By employing an above-described improved sheathed catheter, and by eliminating several items of equipment from a conventional catheterization kit, decreased nursing catheterization time is required and the risk for urinary tract infection is uncompromised or reduced. These and other embodiments, features and potential advantages of the disclosed embodiments will become apparent with reference to the following description and drawings.

### BRIEF DESCRIPTION OF THE DRAWINGS

FIG. 1A is a side view of a sheathed catheter according to an embodiment of the invention.

FIG. 1B is a side view of the sheathed catheter of FIG. 1A, with the sheath partially recessed or drawn back exposing the catheter tip.

FIG. 1C is a side view of a sheathed catheter according to another embodiment of the invention, which includes an introducer that serves, together the sheath, to cover the insertable portion of the catheter.

FIG. 2 is a side view of a sheathed catheter according to another embodiment of the invention, having a free end that is outside the sheath.

FIG. 3 is a side view of a sheathed catheter according to one embodiment of the invention, having a sheath closure adjacent to the urine outlet of the catheter.

FIG. 4 is a side view of a sheathed catheter according to another embodiment of the invention, including a sheath closure similar to FIG. 1 with an attached extension or spout.

FIG. 5A is a side view of a sheathed catheter assembly including a urine collection receptacle.

FIG. 5B is a side view of another sheathed catheter assembly with attached urine collection receptacle.

FIG. 5C is a side view of an assembly similar to that of FIGS. 5A and 5B, in which the urine collection receptacle includes a urine outlet tube.

FIG. 6 shows the contents of a catheterization kit according to an embodiment of the present invention.

### DETAILED DESCRIPTION OF THE PREFERRED EMBODIMENTS

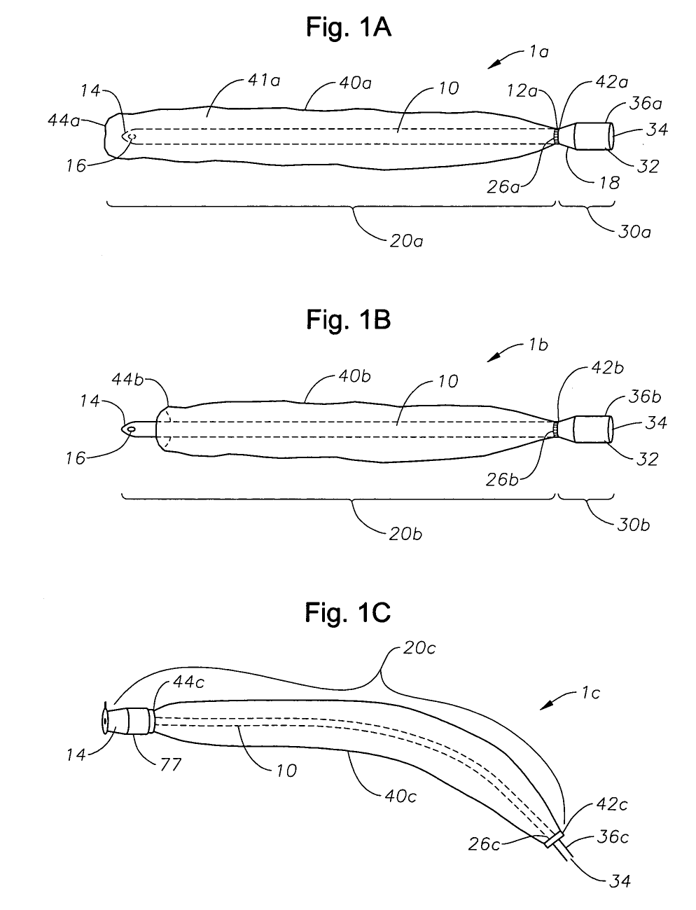

Referring to FIG. 1A, a sheathed catheter assembly 1a is shown. The assembly comprises a flexible catheter 10 comprising a urethra-insertable portion 20a and a portion 30a that is not insertable into the body of the patient. A pliable sheath 40a encloses all, or at least part of, the urethra-insertable portion 20a within lumen 41a, and has a terminus 42a attached to catheter 10 at an attachment point 12a between tip 14 and a second end 32 of the catheter. Second end 32 comprises a urine outlet 34. The urethra-insertable portion 20a commences at the tip end of the catheter 14, which contains one or more urine inlet(s) (16), and ends near sheath attachment point 12a on catheter 10, adjacent to the non-insertable portion 30a. The catheter 10 may be similar to any conventional flexible catheter, e.g., vinyl, red rubber latex, silicone elastomer. The urethra-insertable portion of the catheter is the length of catheter that may be appropriately inserted into the patient's urethra in order to perform a successful catheterization to drain the patient's urinary bladder. The actual length of the catheter, and the length of the insertable portion, will depend on the type of catheter used (e.g., adult or child size) and will also vary somewhat from one patient to another. The insertable portion may terminate at a flared or increased diameter portion 18 of the catheter, which includes end 32 and outlet 34. As a practical matter, the length of catheter available for insertion (insertable portion 20a) is decreased, at least to some extent, by the distance on the catheter that is required to accommodate the collapsed or compressed sheath at terminus 42a, when the catheter assembly is used for its intended purpose. As discussed below in the section titled “Catheterization Procedure,” the sheath collapses or compresses as it is drawn back while advancing the catheter, during use of the assembly. Thus it can be readily appreciated that, in some configurations of the sheathed catheter assembly, less than all of the catheter that is protected by the sheath will actually be accessible and/or needed for insertion into the patient. Accordingly, the insertion stop location 26a (where the catheter stops at the urethral opening upon commencement of urine flow) may substantially coincide with attachment point 12a, or it may be spaced away from attachment point 12b closer to the tip 14. In some embodiments, part of the non-insertable portion of the catheter (e.g., flare 18) is also enclosed by the sheath, as shown in FIG. 3, for example.

The insertable portion 20a of the catheter is maintained in sterile condition inside the protective sheath, which may be similar to an elongated, thin plastic bag. The tip end of the catheter 14 may be initially (i.e., prior to using) fully enclosed by the sheath (FIG. 1A). The sheath may be initially closed and capable of being opened or ruptured so that the catheter tip can emerge from the sheath, when needed. In some embodiments, the sheath terminus 42a and attachment point 12a are positioned at the junction of the insertable and non-insertable portions of catheter 10. The non-insertable portion of the catheter preferably includes a “free end” that is outside of the sheath, as illustrated in FIGS. 1-5.

Referring now to FIG. 1B, an alternative assembly 1b is shown in which sheath 40b is initially open or partially open at terminus 44b. The sheath 40b may also be initially recessed, allowing the tip portion 14 (e.g., 1-2 inches) of catheter 10 to protrude from the sheath. In some applications, this configuration may be preferred over the otherwise similar assembly of FIG. 1A, to facilitate application of a lubricant.

A sheath may either completely enclose the insertable portion of the catheter (FIG. 1A), or attach to and act in cooperation with, an introducer (as shown in catheter assembly 1c in FIG. 1C), or with a catheter tip cover, to keep at least the insertable portion of the catheter sterile prior to and during use. The tip cover or introducer may be similar to those that are known in the art. For example, the O'Neil cover with “cross-cut” end, as described in U.S. Pat. No. 4,652,259 may be attached to the sheath at terminus 44c. Another suitable tip cover or introducer that can be employed with a sheath and catheter is described in U.S. Pat. No. 6,090,075. The disclosures of those patents are hereby incorporated herein by reference.

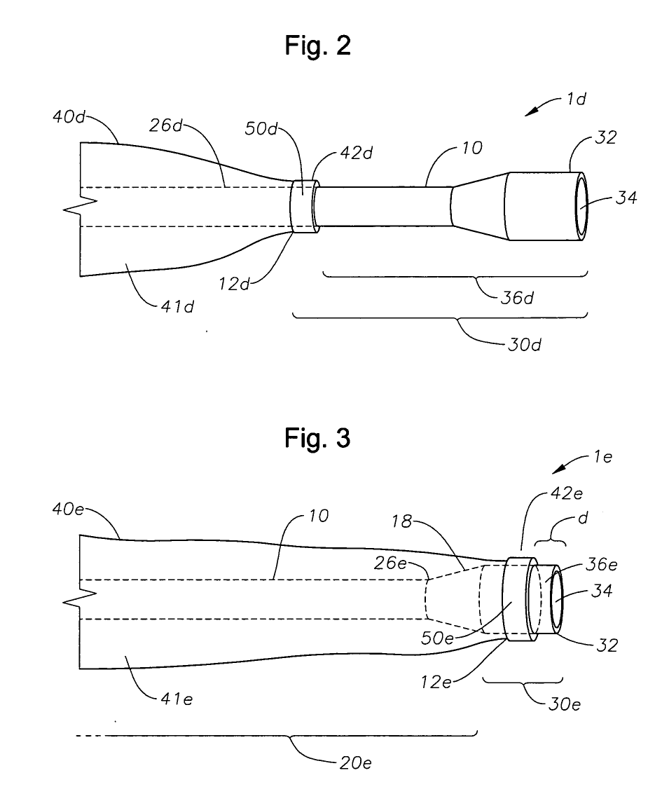

Referring again to the representative embodiment shown in FIG. 1A, terminus 42a of sheath 40a is attached to catheter 10 at an attachment point 12a in such a way that it forms a water-tight or urine-tight seal, preventing entry of liquid or solid contaminants into the sheath interior or lumen 41a. For instance, at terminus 42a the sheath may be gathered and compressed against catheter 10 at attachment point 12a. A closure member such as a plastic or elastomeric tie or compressive band may be employed for attaching the sheath terminus to the catheter, as closure member 50d shown in FIG. 2. Alternatively, the sheath terminus may be attached to the catheter by heat sealing, or with an adhesive, or a combination of adhesive and a tie or band, or any other suitable method that is known in the art. The sheath may be tapered at terminus 42a, to reduce the mass of sheath material to be compressed and attached to catheter 10. Although it is preferred that the sheath terminus 42a-i (FIGS. 1-5) is fixedly attached to the catheter, it could instead be slidingly attached to the catheter for convenience in lengthening the free end, for example, to facilitate positioning the drainage end of the catheter in a receptacle.

With reference now to FIG. 1C, an alternative sheathed catheter assembly 1c is shown, which includes an introducer 77. Catheter tip 14 initially rests inside, and is supported by, introducer 77. Sheath 40c is attached to introducer 77 at terminus 44c, opposite terminus 42c. Together, introducer 77 and sheath 40c cooperate to enclose and protect catheter 10 from contamination prior to and during use. The urethra-insertable portion 20c commences at the tip end 14 of the catheter, and ends near sheath terminus 42c, adjacent to uncovered region 36c of non-insertable portion 30c that is not enclosed by the sheath 40c. A lubricant or a wetting agent is preferably included inside the sheath and/or introducer, to lubricate the catheter in any of the assemblies shown in FIGS. 1-5.

Referring to FIG. 2, a partial view of a sheathed catheter assembly 1d is shown. Preferably the catheter tip/sheath configuration shown in FIGS. 1A-B, or the catheter tip/introducer/sheath combination shown in FIG. 1C, is employed with the end portion shown in FIG. 2. Alternatively, the leading portion of the catheter assembly (not shown in FIG. 2) may be any other suitable configuration for performing a catheterization, provided that it is compatible with the configuration shown in FIG. 2. The sheath terminus is attached to the catheter in the same way as described above with respect to FIGS. 1A-B.

Assembly 1d comprises a flexible catheter 10 comprising a non-insertable portion 30d. In this variation of the catheter assembly, sheath attachment point 12d essentially establishes a boundary for the insertable portion of catheter 10. When an identical catheter 10 is employed in the assembly of FIGS. 2 and 1A, it can be readily seen that attachment point 12d is closer to the catheter tip than attachment point 12a is in FIG. 1A. The distance along the catheter from the tip to attachment point 12d is preferably at least ⅔ of the total length of the catheter. In any case, the selected length of the urethra-insertable portion of the catheter is adequate to reach the bladder of the patient, and to extend into the bladder about 0.5 inch (about 12 mm). Therefore, the distance from tip 14 to insertion stop location 26a could be the same as the distance to insertion stop location 26d for a given patient. For pediatric use, a catheter that has a smaller diameter than a conventional adult size catheter would be employed when constructing the sheathed catheter assembly. For example, pediatric catheters are typically 6-10 French instead of 14 French. The length may be shorter or the same as for adult use. The remaining length of catheter, preferably comprising up to about ⅓ of the length of catheter 10, comprises an uncovered region 36d of non-insertable portion 30d, which is not enclosed by the sheath 40d. This “uncovered region” is sometimes also referred to herein as the “free end” of the catheter, and in the embodiment of FIG. 2, comprises most of the length of the non-insertable portion 30d. Otherwise, this embodiment is preferably similar to FIG. 1A, and includes a sheath 40d having a lumen 41d and terminus 42d that is attached to the catheter at attachment point 12d, by closure 50d, for example. Insertion stop location 26d in this configuration is more distant from catheter end 32 than that shown in FIG. 1A. In some embodiments of this catheter assembly, the free end 36d is no more than about ⅓ of the length of the catheter, to allow for an adequate length of insertable catheter, and to provide an easily manipulated free end to provide an area adjacent to the urine outlet that can rest on the opening of a urine collection tray, or which can be attached to a urine collection bag. In some circumstances, it is necessary to have this ⅓ length of the catheter free, allowing manipulation of the urine discharge end to quickly reposition it between the urine collection receptacle and a separate urine collection container. A longer free end is preferably avoided, however, in order to keep the free end from dipping into the urine receptacle and possibly introducing contaminants. A longer free end also reduces the insertable length of the catheter that is available for use, i.e., the distance from the catheter tip to the insertion stop location 26d. Preferably, the sheathed catheter assembly includes a free end about 5 mm cm to about 50 mm long, more preferably in the range of about 10 mm to about 30 mm. A free end that extends more than about 2 inches (about 50 mm) beyond the sheath attachment point 12 is considered sub-optimal.

As shown in FIG. 3, another variation of the catheter assembly 1e comprises sheath 40e having a lumen 41e enclosing all or part of the insertable portion 20e, and a portion of the non-insertable portion 30e of catheter 10. Terminus 42e of sheath 40e is attached via closure 50e to attachment point 12e close to the end 32 of catheter 12, thereby leaving little or no “free end” 36e of the catheter. Similar to the configuration shown in FIGS. 1A and 1B, the insertion stop location 26e and the insertable portion 20e are limited by the flared structure 18 adjacent to end 32. In the embodiment shown in FIG. 3, the flare 18 provides a convenient space for the gathered folds of sheath to occupy, and optimizes the length of catheter available for insertion. Thus, insertion stop location 26e is illustrated as coinciding with the beginning of flare 18, in FIG. 3. In similar embodiments in which the sheathed catheter assembly has no free end, or substantially no free end, the sheath attachment point essentially coincides with end 32 of catheter 10, or it may be merely 1 mm to a few millimeters back from the outlet 32. For instance, a free end 36e of only about 2 mm is adequate in many situations of use to facilitate urine collection in a conventional disposable tray, and to facilitate catching a sterile urine specimen in a conventional specimen container. When there is no free end, the sheath attachment point preferably serves the above-described purpose of resting stably on the urine receptacle while the bladder drains, or it provides a convenient grasping area for attaching the outlet 34 to a collection bag, or for cooperating with another urine disposal means. To facilitate those purposes, a tapered sheath may be preferred.

A feature of many of the above-described variations of the sheathed catheter assembly, is that, regardless of the chosen configuration of the catheter tip end of the assembly, at least the urethra-insertable portion of the catheter is covered, to prevent contamination and urinary tract infections. In some variations, the catheter tip end of the assembly initially protrudes from the sheath, to facilitate application of a lubricant gel. An advantageous feature of each embodiment of the catheter assembly is that the sheath does not extend beyond the urine outlet end 32 of the catheter 10, leaving end 32 free to allow urine to drain out into the tray for accurate measurement and/or to drain into a urine collection container. This configuration ensures that no urine will enter the sheath interior or lumen, and that urine and other possible contaminants cannot contact the insertable portion of the catheter. Preferably a lubricant, such as K-Y Jelly™ or a wetting agent is contained inside the sheath and/or in a catheter tip cover, or the like, if provided. The sheath/catheter attachment advantageously prevents the lubricant or wetting agent from mixing with the urine and possibly skewing urinalysis results.

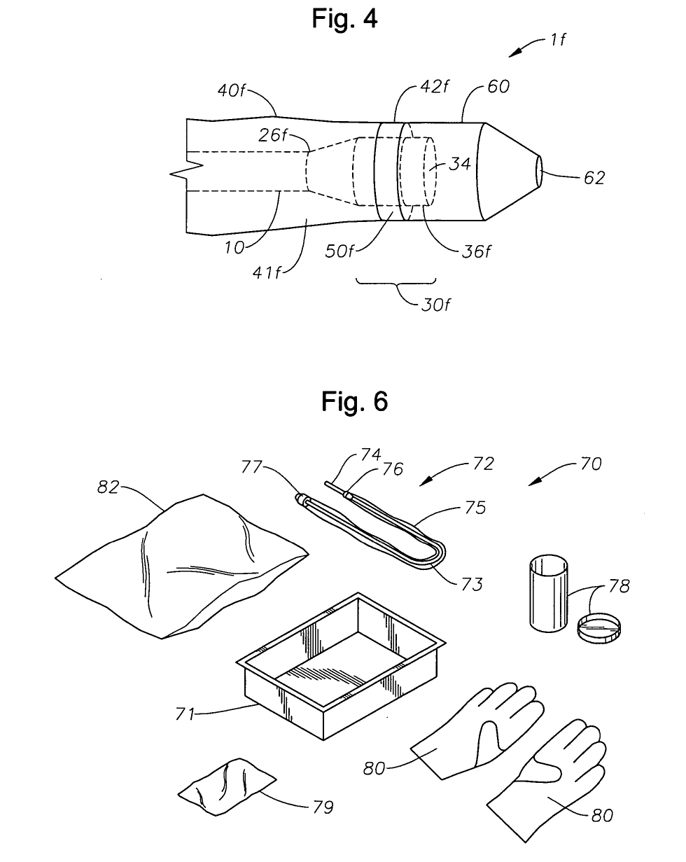

In FIG. 4, a modified version of the assembly of FIG. 3 is shown, comprising catheter 10 with outlet 34 and insertion stop location 26f, and sheath 40f having lumen 41f. In this configuration, the assembly 1c also includes an extension or pour spout 60, with urine outlet 62, to facilitate obtaining a sterile urine specimen from the catheter outlet 34. Extension 60 may be formed together with closure or band 50f, or it may be provided separately and snapped over band 50f and held in place by friction fit, or other suitable attachment method. Free end 36f is outside sheath 40f, but is enclosed in extension 60. In some situations, extension 60 may be preferred for resting on the edge of the collection container to drain the exiting urine.

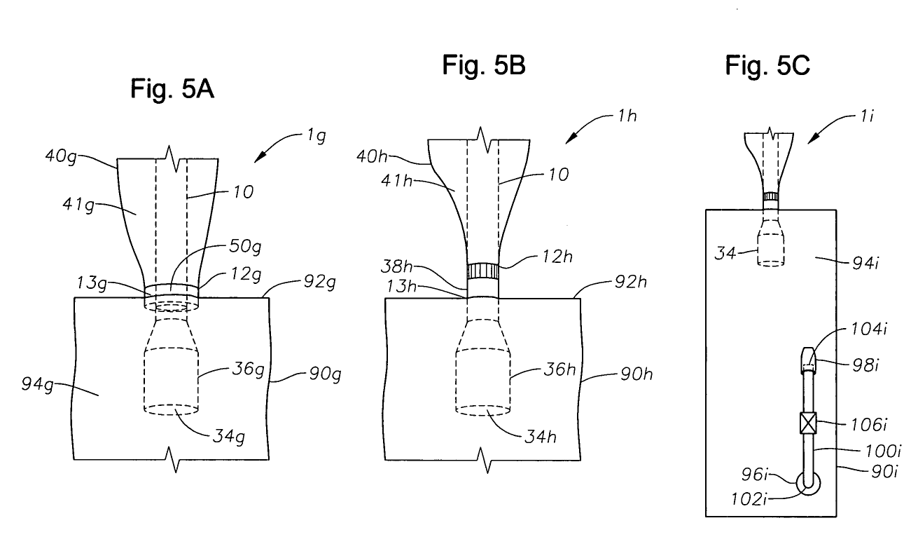

Referring now to FIG. 5A, another embodiment of the sheathed catheter assembly includes an attached urine collection receptacle. The urine receptacle or bag is preferably made of a flexible, water-proof material (e.g., plastic) and is sized to hold a volume of urine in the range of 700-2000 mL, preferably about 1000 mL. The urethra-contacting end of the assembly may be of any suitable configuration. Free end 36g of catheter 10 is similar to any of those illustrated in FIGS. 1A-C and 3, and described above. In representative assembly 1g in FIG. 5A, bag 90 has a terminus 92g which is attached to catheter 10 at bag attachment point 13g, which, in this instance, substantially coincides with attachment point 12g (of sheath 40g) on catheter 10. A collar or band 50g may be included to secure the water-tight attachment of sheath 40g, collection bag 90 and catheter 10. In this assembly, the free end 36g, having a urine outlet 34, is inside the lumen 94g of urine receptacle 90g. Lumen 94g is not in fluid communication with lumen 41g of sheath 40g.

The design of assembly 1g in FIG. 5A may be varied somewhat, as shown in FIG. 5B. Assembly 1h is configured so that a portion of the free end is not enclosed in urine collection bag 90h. In this embodiment, section 38h of free end 36h is exposed (i.e., not enclosed in either sheath 40h or bag 90h), to facilitate manipulation of the free end and of the urine collection bag 90h. Accordingly, bag attachment point 13h is spaced apart from attachment point 12h on catheter 10. Still another representative catheter assembly 1i, with attached urine collection bag 90i, is shown in FIG. 5C. A drainage tube 100i is attached at end 102i to bag 90i at drainage port 96i, which is spaced apart from catheter outlet 34. Outlet end 104i of tube 100i is releasably retained in holder 98i on bag 90i, to maintain sterility of end 104i. A closure 106i is attached to tube 100i between ends 102i, 104i and is capable of being operated to either prevent or permit urine flow urine flow from lumen 94i bag 90i to outlet end 104i.

Catheterization Kit

As shown in FIG. 6, a representative catheterization kit (“cath kit”) 70 that is particularly useful for hospital inpatient catheterizations includes a sheathed catheter assembly 72, representative of those described above and shown as 1a-f of FIGS. 1-4. For example, sheathed catheter assembly 72 may include an introducer 77, catheter 73, and sheath 75 attached at one end 76 to the catheter and at the opposite end to the introducer 77, if present. The free end 74 of catheter 73 is not covered by sheath 75. In addition to the sheathed catheter assembly, the kit preferably also contains a packet of antiseptic swabs 79 (e.g., three swabs saturated with Betadine, Povidone-Iodine or other suitable antiseptic), disposable gloves 80, small urine specimen bottle (with cap) 78, and a tray 71 that holds these supplies and also serves as a urine collection container and has a capacity in the range of 700-2000 mL, preferably about 1000 mL. Preferably a fenestrated drape is also included in the kit. A gauze pad may also be included in the kit as a convenient wipe at the end of the procedure. The kit components are protected by a sanitary wrapper or cover 82. All kit components are preferably disposable.

Notably absent from the present kit, however, are the customary liquid antiseptic packet, cotton balls, tray for cotton balls, forceps, and packet containing lubricating jelly. In some embodiments, the fenestrated drape is also omitted from the kit without compromising sterile technique. The conventional waterproof absorbent pad is also unnecessary, as it was often included in the past primarily to provide a sterile field for placement of the sterile jelly. Preferably, the lubricant is provided within the catheter assembly. For example, a lubricating amount of sterile lubricant may be present inside the sheath lumen, or inside an introducer (FIG. 1C). Alternatively, the catheter may be a lubricated hydrophilic type as is known in the art, in which case the lumen and/or an introducer of the catheter assembly may contain an aqueous wetting agent. A drawback of conventional apparatus and methods is that touching the catheter to any surface outside of the sterile field, as when lubricating jelly is applied, for instance, increases the risk of contamination and urinary tract infection. In contrast, with the present kit, if the sheathed catheter touches anything outside the sterile field, the insertable portion of the catheter remains sterile. Another potential problem associated with conventional catheterization kits and procedures is that, if a glove touches anything outside the sterile field and then touches the catheter, the catheter becomes contaminated. If a sheathed catheter assembly is employed instead, and if a glove becomes contaminated, the catheter nevertheless remains sterile inside the protective sheath. Thus, the risk of infection and patient morbidity is reduced or eliminated with use of the new catheterization kit and catheterization procedure.

The above-mentioned items omitted from conventional cath trays are not needed for carrying out a streamlined catheterization procedure with the above-described sheathed catheter assembly and maintaining sterile technique.

Catheterization Procedure

The simplified catheterization kit is preferably employed as follows: After opening the sanitary wrapper 82, the nurse dons the gloves 80 and places the fenestrated drape, if provided, around the patient's genitalia. The packet 79 containing the antiseptic swabs is opened and the urethral area around the urethral opening is cleansed using the antiseptic swabs. Referring to FIG. 1A, the nurse grasps catheter 10 through the soft, flexible sheath 40a near the tip end of the catheter and advances the catheter tip into the patient's urethra. If necessary, the sheath terminus 44a is first opened to allow the catheter tip to emerge. The sheath 40a is pulled back to expose the catheter tip, giving the catheter assembly an appearance similar to that shown in FIG. 1B. As the catheter tip is advanced by the nurse, a portion of sheath 40a becomes gathered or collapses toward sheath attachment point 12a. By appropriately repositioning the nurse's grasp on catheter 10 through sheath 40a, and continuing to gently urge the catheter into the urethra and toward the bladder, the sheath 40a is caused to continue gathering toward sheath attachment point 12a. When the catheter tip enters the patient's bladder a sufficient distance to commence draining of accumulated urine, and advanced into the bladder sufficiently (e.g., about 1 cm) to establish optimal urine flow, further insertion ceases. As a result, the position on catheter 10 that is at the urethral opening when insertion ceases is termed the “insertion stop location” 26a.

A catheter assembly like any of those shown in FIGS. 1C, and 2-4 may be substituted for the catheter assembly shown in FIG. 1A or 1B, and are used similarly. The insertion stop location (insertion stop location 26a in FIG. 1A) on the catheter will vary somewhat in different situations of use. The insertion stop location will also vary somewhat with alternative catheter assembly designs. For instance, in the embodiment shown in FIG. 3, the maximum insertable length of catheter, and the insertion stop location are limited by an expanded diameter or flared portion 18 of the catheter. In FIG. 2, insertion stop location 26d of the insertable portion is in an area between the catheter tip and the sheath attachment point 12d, but is spaced apart from the end 32 of the catheter. Prior to commencement of urine drainage, the free end or recess 36a-e (FIGS. 1-3), or extension 60 (FIG. 4), as applicable, is positioned on the urine receptacle, so that urine can drain into the tray without permitting the catheter to contact the collected urine. Alternatively, any other suitable urine receptacle may be used instead of the disposable tray provided as part of the kit. When employing an embodiment of the catheter assembly that has no “free end” (similar in appearance to FIG. 3, but wherein distance “d” is essentially zero), the attachment point 12e, or closure 50e may be positioned or rested on tray 71 for collection of urine.

After commencement of urine flow, the outlet 34 or spout 62 (FIG. 4) may be directed briefly into the specimen container 78, to collect a sterile specimen, as needed. Upon completion of urine evacuation, catheter 10 or 73 is simply withdrawn from the urethra and disposed of along with the other components of the kit. If desired, the catheter may be retracted into the sheath prior to disposal. The entire catheterization process can usually be accomplished by a nurse in about 5-7 minutes, maintaining sterile technique throughout the procedure.

Because multiple steps are involved in a typical inpatient catheterization, a nurse necessarily spends a significant amount of time (e.g., 10-15 minutes) performing each catheterization. The conventional catheterization kit and sterile procedure that is used in virtually every inpatient hospital around the world has remained essentially the same for 50 years. The new kit and simplified procedure offers a way to simplify and streamline inpatient catheterization procedures without compromising sterile technique. Every item that the new kit eliminates from the customary catheterization setup will decrease the number of procedural steps, and also reduces the amount of nursing time needed. Fewer steps also reduces the patient's risk for urinary tract infection, and decreases inconvenience for the patient. It is estimated that at least 50% less nursing time is required to carry out a catheterization procedure with the new kit.

What is claimed is:

1. A urinary catheter assembly comprising:
a catheter comprising a first end, a second end having a urine outlet, a urethra-insertable portion containing an insertion stop location, and a non-insertable portion;
a pliable sheath comprising a lumen and enclosing all or part of said insertable portion, and having a terminus sealingly attached to said catheter at an attachment point disposed between said insertion stop location and said second end, wherein the distance along said catheter from said attachment point to said second end is no more than about one-third of the total length of the catheter from said first end to said second end, provided that said lumen and said urine outlet are not in mutual fluid communication.

2. The catheter assembly of claim 1 further comprising an introducer attached to said sheath opposite said terminus, said introducer and said sheath together enclosing all of said urethra-insertable portion.

3. The catheter assembly of claim 1, wherein at least part of said non-insertable portion comprises an uncovered region of said catheter outside said sheath.

4. The catheter assembly of claim 1 wherein said attachment point coincides with said second end or is spaced apart from said second end a distance (d) of no more than about 2 mm.

5. The catheter assembly of claim of claim 1 wherein said attachment point is spaced apart from said second end a distance (d) in the range of about 2 mm to about 50 mm.

6. The catheter assembly of claim 5 wherein said distance (d) is in the range of about 10 mm to about 30 mm.

7. The catheter assembly of claim 1 wherein said sheath terminus is sealingly attached to said attachment point on said catheter.

8. The catheter assembly of claim 1 wherein said sheath terminus is slidingly attached to said attachment point on said catheter.

9. The catheter assembly of claim 1 further comprising a urine receptacle attached to said non-insertable portion of said catheter.

10. The catheter assembly of claim 9 wherein said non-insertable portion comprises an uncovered section disposed between said sheath and said urine receptacle.

11. The catheter assembly of claim 9 wherein said urine receptacle comprises a urine drainage tube.

12. A urinary catheter assembly comprising:
a catheter comprising a first end, a second end having a urine outlet, a urethra-insertable portion containing an insertion stop location, and a non-insertable portion;
a pliable sheath comprising a lumen and enclosing all or part of said insertable portion and having a terminus sealingly attached to said catheter at an attachment point disposed between said insertion stop location and said second end; and
an extension attached to said sheath terminus, said extension having a urine spout, provided that said lumen and said urine outlet are not in mutual fluid communication.

13. A urinary catheter assembly comprising:
a catheter comprising a first end, a second end having a urine outlet, a urethra-insertable portion containing an insertion stop location, and a non-insertable portion;
a pliable sheath comprising a lumen and enclosing all or part of said insertable portion and having a terminus slidingly attached to said catheter at an attachment point disposed between said insertion stop location and said second end, provided that said lumen and said urine outlet are not in mutual fluid communication.

14. A catheterization kit comprising:
the urinary catheter assembly of claim 1; and
a sanitary wrapper enclosing said assembly.

15. The kit of claim 14 consisting essentially of the following items:
said urinary catheter assembly;
at least one antiseptic swab;
disposable gloves;
a urine specimen container; and
a tray capable of holding said catheter assembly, swab(s), gloves, and specimen container, wherein said tray is also capable of serving as a urine receptacle; and a sanitary wrapper enclosing said items.

16. The kit of claim 14 excluding one or more drapes.

17. The kit of claim 14 excluding forceps.

18. The kit of claim 14 excluding antiseptic liquid.

19. A catheterization kit comprising:
the urinary catheter assembly of claim 12; and
a sanitary wrapper enclosing said assembly.

20. A catheterization kit comprising:
the urinary catheter assembly of claim 13; and
a sanitary wrapper enclosing said assembly.
